(19) United States
(12) Patent Application Publication
(10) Pub. No.: US 2021/0161371 A1
(43) Pub. Date: Jun. 3, 2021

(54) ENDOSCOPE

(71) Applicant: OLYMPUS CORPORATION, Tokyo (JP)

(72) Inventor: Nobuaki AKUI, Tokyo (JP)

(73) Assignee: OLYMPUS CORPORATION, Tokyo (JP)

(21) Appl. No.: 17/177,345

(22) Filed: Feb. 17, 2021

Related U.S. Application Data
(63) Continuation of application No. PCT/JP2018/030811, filed on Aug. 21, 2018.

(51) Int. Cl. A61B 1/018; A61B 1/005; A61B 18/22

(52) U.S. Cl. CPC A61B 1/018; A61B 1/0052; A61B 18/22; A61B 1/0057

(57) ABSTRACT

An endoscope includes an insertion section including a bending portion configured to bend in response to pulling and relaxing of a pair of wires, an operation section, a pulling mechanism configured to pull and relax the pair of wires, a holding portion holding a treatment member, and a driving member configured to move the holding portion in a direction along a longitudinal axis of the treatment member. The pulling member includes a pulley to which the pair of wires is connected, a shaft member protruding from the pulley and having a central axis coinciding with a central axis of the pulley, and a support portion supporting the shaft member such that the shaft member is rotatable about the central axis, and configured to allow a movement of the shaft member in a first direction and a second direction that are parallel to the central axis of the shaft member.

### CROSS REFERENCE TO RELATED APPLICATION

This application is a continuation application of PCT/JP2018/030811 filed on Aug. 21, 2018, the entire contents of which are incorporated herein by this reference.

### BACKGROUND OF THE INVENTION

### 1. Field of the Invention

The present invention relates to an endoscope including an insertion section that is provided with a portion configured to actively bend.

### 2. Description of the Related Art

Medical endoscopes are provided with an elongated insertion section insertable into a subject, thereby allowing observation of the inside of the subject and treatment using an instrument such as a treatment instrument. An endoscope for renal pelvis/urinary organs (pyeloureteroscope) disclosed, for example, in Japanese Patent Application Laid-Open Publication No. 2016-187554 is known as such a medical endoscope.

Examples of treatment using an endoscope for renal pelvis/urinary organs include treatment for removing a urinary stone. In this treatment, an optical fiber cable is caused to protrude from an insertion section of the endoscope, and a stone is broken up by irradiating the stone with laser light using the optical fiber cable.

When performing this treatment, a user of the endoscope irradiates a stone with laser light while operating both the endoscope and the optical fiber cable at the same time. More specifically, when performing this treatment, the user of the endoscope performs, with one hand, an operation of bending an insertion section and an operation of rotating the insertion section about a longitudinal axis while holding the operation section. At the same time, the user of the endoscope performs, with the other hand, an operation of advancing and retracting the insertion section in a direction along the longitudinal axis and an operation of advancing and retracting the optical fiber cable in the direction along the longitudinal axis.

### SUMMARY OF THE INVENTION

An endoscope according to an aspect of the present invention includes: an insertion section configured to be inserted into a subject, and including a bending portion configured to bend in a predetermined direction in response to pulling and relaxing of a pair of wires; an operation section connected to a proximal end side of the insertion section; a pulling mechanism to which the pair of wires is connected, the pulling mechanism being disposed at the operation section and configured to pull and relax the pair of wires; a holding portion disposed at the operation section, and holding a treatment member inserted in a channel provided inside the insertion section; and a driving member disposed at the operation section, and configured to move the holding portion in a direction along a longitudinal axis of the treatment member, wherein the pulling mechanism includes a pulley to which the pair of wires is connected, a shaft member protruding from the pulley, and having a central axis coinciding with a central axis of the pulley, and a support portion supporting the shaft member such that the shaft member is rotatable about the central axis, and configured to allow a movement of the shaft member in a first direction and a second direction that are parallel to the central axis of the shaft member.

### BRIEF DESCRIPTION OF THE DRAWINGS

FIG. 1 is a diagram illustrating a schematic configuration of an endoscope;

FIG. 2 is a diagram illustrating a schematic configuration of the endoscope;

FIG. 3 is a diagram illustrating a pulling mechanism portion and wires;

FIG. 4 is a cross-sectional view taken along line IV-IV of FIG. 3, illustrating a shaft member located at an end of a movable range in a first direction;

FIG. 5 illustrates the shaft member located at an end of the movable range in a second direction, in a cross section of FIG. 4;

FIG. 6 is a diagram illustrating a modification of an operation lever; and

FIG. 7 is a diagram illustrating a modification of a driving portion.

### DETAILED DESCRIPTION OF THE PREFERRED EMBODIMENTS

A preferred embodiment of the present invention will now be described with reference to the drawings. Note that in each of the drawings to be referred to in the following description, the scale varies between components such that each of the components is large enough to be recognizable in the drawings. The present invention is not limited only to the embodiment illustrated in the drawings in terms of the quantity of the components, the shape of the components, the size ratio of the components, and the relative positional relationship between the individual components illustrated in the drawings.

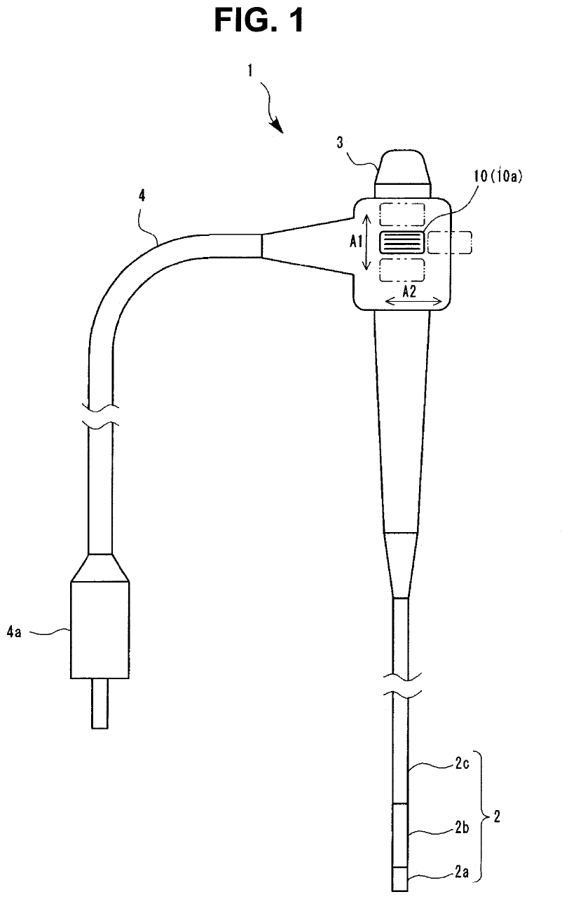

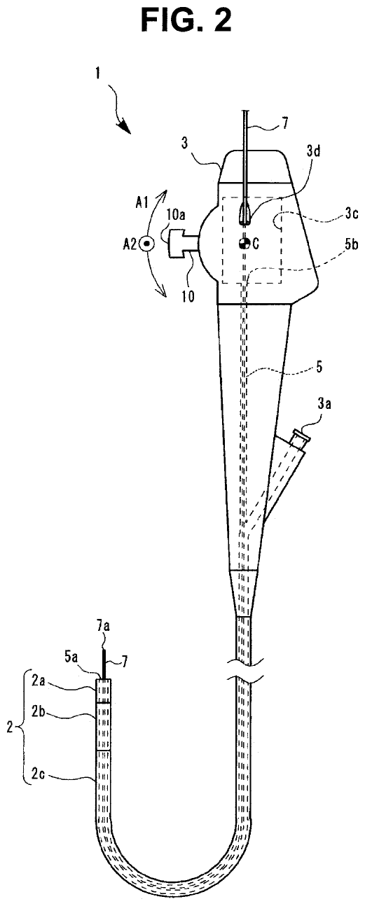

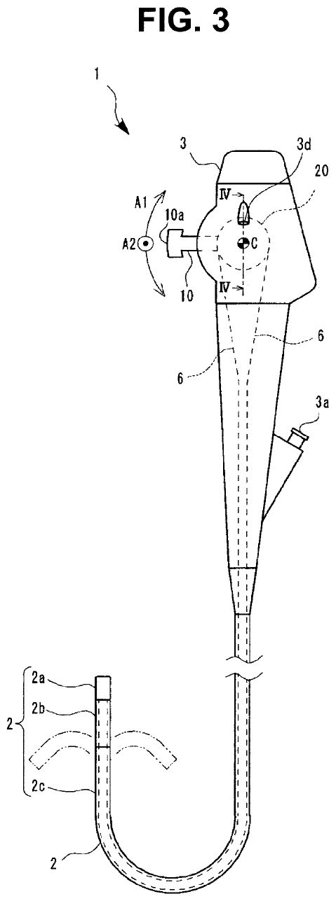

FIG. 1 is a diagram illustrating a schematic configuration of an endoscope. FIG. 2 is a diagram illustrating a channel provided in the endoscope. FIG. 3 is a diagram illustrating a pulling mechanism portion and wires provided in the endoscope.

An endoscope 1 illustrated in FIG. 1 includes an elongated insertion section 2 insertable into a subject. The insertion section 2 has a configuration that allows observation of the inside of the subject. In the present embodiment, the subject is a human body, for example. In the present embodiment, the endoscope 1 is an endoscope for renal pelvis/urinary organs (pyeloureteroscope), for example. In the present embodiment, the endoscope 1 is embodied as a so-called videoscope, for example.

The endoscope 1 includes the elongated insertion section 2 formed along a longitudinal axis, an operation section 3 located at one end, namely, a proximal end of the insertion section 2, and a universal cord 4 extending from the operation section 3. In the following description, the other end of the insertion section 2 opposite to the proximal end will be referred to as a distal end.

The universal cord 4 includes a connector 4a connected to an external device (not illustrated).

The operation section 3 is a section held by a user. The operation lever 10 is disposed at the operation section 3. A pulling mechanism portion 20, a holding portion 30, and a driving portion 40 are disposed at the operation section 3 (not illustrated in FIGS. 1 and 2).

The operation lever 10 is a member that allows the user to operate the pulling mechanism portion 20 and the holding portion 30 to be described below. The operation lever 10 is exposed to an outer surface of the operation section 3. The operation lever 10 is movable relative to the operation section 3 along a first axis A1 and a second axis A2 intersecting the first axis A1. The way the operation lever 10 moves relative to the operation section 3 is not particularly limited. The operation lever 10 may move along a straight line, or may move along a curve.

In the present embodiment, as illustrated in FIGS. 1 and 2, a distal end portion 10a of the operation lever 10 protrudes from the outer surface of the operation section 3, for example. The distal end portion 10a of the operation lever 10 is disposed at such a position that, for example, when the user holds the operation section 3, the thumb of the hand holding the operation section 3 reaches the distal end portion 10a.

The operation lever 10 rotates about a predetermined rotation axis C, relative to the operation section 3. The operation lever 10 also moves linearly, parallel to the rotation axis C, relative to the operation section 3. In the present embodiment, when the operation lever 10 rotates about the rotation axis C, the distal end portion 10a of the operation lever 10 moves along the first axis A1. When the operation lever 10 moves parallel to the operation section 3, the distal end portion 10a moves along the second axis A2. Accordingly, the first axis A1 is a curve with a predetermined curvature, and the second axis A2 is a straight line. In other words, when the user moves the distal end portion 10a along the first axis A1, the operation lever 10 rotates about the rotation axis C. When the user moves the distal end portion 10a along the second axis A2, the operation lever 10 moves parallel to the rotation axis C.

In the present embodiment, when the user holds the operation section 3 such that the insertion section 2 extends downward and such that the operation lever 10 faces the user, a vertical direction as viewed from the user is a direction along the first axis A1, and a horizontal direction is a direction along the second axis A2, for example.

The configuration for supporting the operation lever 10 in such a manner that the operation lever 10 is movable relative to the operation section 3, and the configuration of the pulling mechanism portion 20, the holding portion 30, and the driving portion 40, which are disposed inside the operation section 3, will be described below.

As illustrated in FIG. 2, the operation section 3 has a treatment instrument insertion port 3a, and a second opening 5b. The treatment instrument insertion port 3a and the second opening 5b communicate with a channel 5 to be described below. In the present embodiment, an optical fiber cable 7 configuring a treatment member can be inserted into the channel 5 through the second opening 5b. The optical fiber cable 7 is a treatment instrument that irradiates a stone with laser light inside the subject.

The treatment instrument insertion port 3a is open on the outer surface of the operation section 3. In the present embodiment, an internal space 3c is formed in the operation section 3, for example. The second opening 5b is open on an inner wall surface of the internal space 3c. A through hole 3d communicating with the internal space 3c is formed in the outer surface of the operation section 3. The optical fiber cable 7 is inserted into the through hole 3d.

The insertion section 2 includes a distal end portion 2a, a bending portion 2b, and a flexible tube portion 2c. The distal end portion 2a, the bending portion 2b, and the flexible tube portion 2c are connected in this order from the distal end toward the proximal end, along a longitudinal axis of the insertion section 2.

The distal end portion 2a is disposed at the distal end of the insertion section 2. The distal end portion 2a has a first opening 5a. Although not illustrated, an image pickup unit and an illumination light emitting portion are disposed at the distal end portion 2a.

The first opening 5a communicates with a distal end of the channel 5 provided inside the insertion section 2 and the operation section 3. As described above, the channel 5 communicates with the treatment instrument insertion port 3a and the second opening 5b provided in the operation section 3.

The treatment instrument inserted into the channel 5 through the treatment instrument insertion port 3a or the second opening 5b protrudes from the first opening 5a. In the present embodiment, a distal end 7a of the optical fiber cable 7 inserted into the channel 5 through the second opening 5b protrudes from the first opening 5a.

The image pickup unit is a device for picking up an optical image. The image pickup unit includes an objective lens and an image pickup device. Note that part of the image pickup unit may be disposed in the operation section 3. For example, the image pickup unit may include an objective lens disposed at the distal end portion 2a, an image pickup device disposed in the operation section 3, and an image guide fiber inserted in the insertion section 2.

The image pickup unit is electrically connected to the connector 4a, through an electrical cable disposed in the endoscope 1. When the connector 4a is connected to an external device, the image pickup unit is electrically connected to the external device. The external device includes a processor that displays an optical image picked up by the image pickup unit on a display device (not illustrated).

The illumination light emitting portion emits light that illuminates an object of the image pickup unit. The light source of the light emitted from the illumination light emitting portion may be disposed in the endoscope 1, or may be disposed in an external device. Techniques for the image pickup unit and the illumination light emitting portion of the endoscope 1 are well known, and will not be described in detail.

The bending portion 2b actively bends in response to an operation of the operation lever 10 by the user. In the following description, a neutral state refers to a state in which a longitudinal axis of the bending portion 2b is straight. The bending portion 2b bends in two mutually opposing directions from the neutral state.

As illustrated in FIG. 3, a pair of wires 6 is inserted in the insertion section 2. A distal end of each of the wires 6 is fixed near a distal end of the bending portion 2b. The bending portion 2b is flexible, and the bent shape varies according to the amount of pulling the pair of wires 6 in a proximal end direction. Note that the configuration of the bending portion 2b having a bent shape that varies according to the amount of pulling the pair of wires 6 inserted in the insertion section 2 is the same as the configuration of a well-known endoscope, and will not be described herein.

A proximal end of the pair of wires 6 is connected to the pulling mechanism portion 20 disposed in the operation section 3. The pulling mechanism portion 20 configuring a pulling mechanism pulls and relaxes the pair of wires 6, in response to an operation of the operation lever 10 by the user.

The flexible tube portion 2c is disposed on a proximal end side of the bending portion 2b. A proximal end of the flexible tube portion 2c is connected to the operation section 3. The flexible tube portion 2c is flexible, and passively bends in response to an external force.

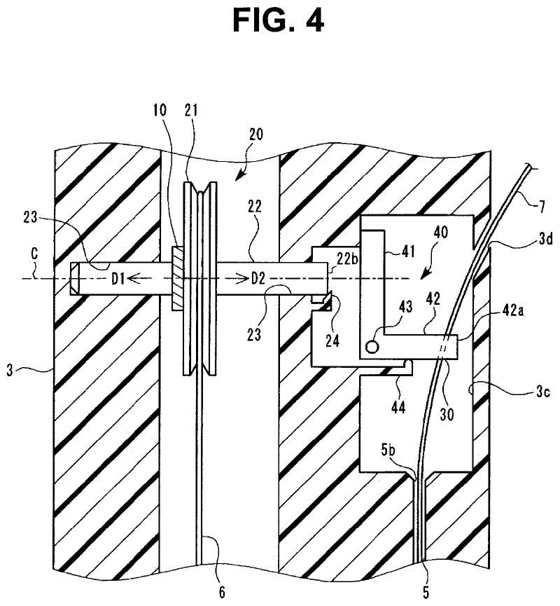

The configuration of the pulling mechanism portion 20, the holding portion 30, and the driving portion 40 disposed in the operation section 3 will now be described. FIG. 4 is a cross-sectional view taken along line IV-IV of FIG. 3. FIG. 4 illustrates a cross section in a plane including the rotation axis C of the operation lever 10.

As illustrated in FIG. 4, the pulling mechanism portion 20 includes a pulley 21, a shaft member 22, and a support portion 23. The pulley 21 is a disc-shaped member. The proximal end of the pair of wires 6 is connected to a cylindrical outer peripheral surface of the pulley 21. The individual wires 6 are wound in different directions around the outer peripheral surface of the pulley 21.

The shaft member 22 is a columnar member protruding from the pulley 21, and is disposed such that a central axis of the shaft member 22 coincides with a central axis of the pulley 21. The shaft member 22 may be formed as a separate member from the pulley 21, or may be formed as an integral member with the pulley 21. In the present embodiment, the shaft member 22 includes a pair of columns protruding parallel to the central axis of the pulley 21 from opposite end faces of the pulley 21, for example. In the following description, one of directions parallel to the central axis of the shaft member 22 (pulley 21) is referred to as a first direction D1, and the other is referred to as a second direction D2.

The support portion 23 is a pair of bearings supporting the shaft member 22 such that the shaft member 22 is rotatable about the central axis with respect to the operation section 3. The support portion 23 allows a movement of the shaft member 22 in a direction parallel to the central axis. In the present embodiment, the support portion 23 includes a pair of holes provided in the operation section 3, for example. The pair of columns of the shaft member 22 respectively protruding in the first direction and the second direction from the pulley 21 is inserted in the pair of holes of the support portion 23. In other words, the support portion 23 of the present embodiment is a pair of sliding bearings.

As illustrated in FIG. 4, the support portion 23 supporting a first direction side of the shaft member 22 communicates with the internal space 3c. A distal end portion 22b defining an end of the shaft member 22 in the second direction D2 protrudes into the internal space 3c through the support portion 23. When the shaft member 22 moves parallel to the central axis with respect to the operation section 3, the amount by which the distal end portion 22b of the shaft member 22 protrudes into the internal space 3c changes.

The support portion 23 includes a stopper 24 that locks the shaft member 22 at an end in the first direction D1, in a movable range in a direction parallel to the central axis. The stopper 24 allows a movement of the shaft member 22 in the second direction D2 when a force in the second direction D2 applied to the shaft member 22 exceeds a predetermined value.

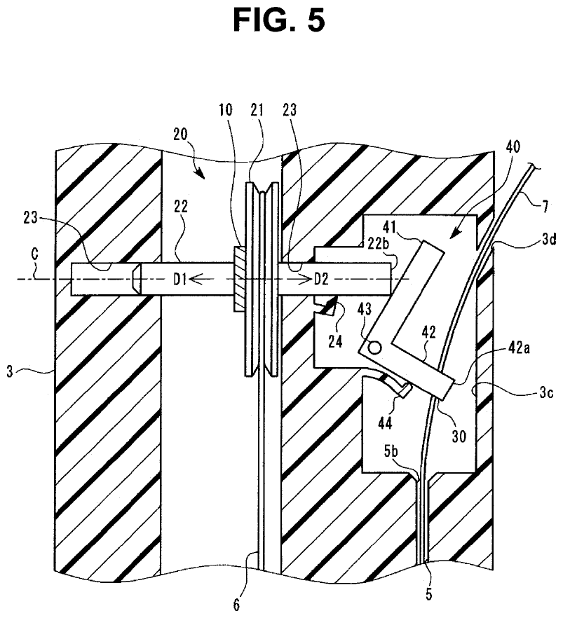

FIG. 4 illustrates the shaft member 22 located at the end of the movable range in the first direction D1. FIG. 5 illustrates the shaft member 22 located at an end of the movable range in the second direction D2.

The stopper 24 of the present embodiment is made of an elastically deformable material. The stopper 24 is a protrusion protruding from an outer side of an outer circumference of the shaft member 22 toward the central axis. As illustrated in FIG. 4, when the shaft member 22 is located at the end of the movable range in the first direction D1, the stopper 24 is located on the inner side of (at a position closer to the central axis than) the outer circumference of the shaft member 22. Accordingly, in this case, when the distal end portion 22b of the shaft member 22 comes into contact with the stopper 24, the movement of the shaft member 22 in the second direction D2 is restricted.

The stopper 24 is elastically deformed so as to be pushed away by the shaft member 22, by a force in the second direction D2 applied to the shaft member 22. More specifically, the stopper 24 is moved in a direction away from the central axis of the shaft member 22, by a force in the second direction D2 applied to the shaft member 22. When a force in the second direction D2 applied to the shaft member 22 exceeds a predetermined value, the stopper 24 moves to the same position as the position of the outer circumference of the shaft member 22 from the central axis as illustrated in FIG. 5. In this case, the stopper 24 allows a movement of the shaft member 22 in the second direction D2.

Note that the stopper 24 of the present embodiment is disposed at a distal end of a plate spring protruding from the inner wall surface of the internal space 3c of the operation section 3. In the present embodiment, the plate spring and the stopper 24 are integrally molded with a resin member configuring the inner wall surface of the internal space 3c.

In the present embodiment, a proximal end of the operation lever 10 is fixed to the shaft member 22 or the pulley 21. In other words, the rotation axis C of the operation lever 10 coincides with the central axes of the shaft member 22 and the pulley 21.

Therefore, according to the endoscope 1 of the present embodiment, when the distal end portion 10a of the operation lever 10 moves along the first axis A1, the shaft member 22 or the pulley 21 rotates about the central axis (rotation axis C). In other words, when the user swings the operation lever 10 in the vertical direction, the shaft member 22 or the pulley 21 rotates about the central axis.

According to the endoscope 1 of the present embodiment, when the distal end portion 10a of the operation lever 10 moves along the second axis A2, the shaft member 22 or the pulley 21 moves in the first direction D1 or the second direction D2 parallel to the central axis. When the user moves the operation lever 10 to the left, the shaft member 22 or the pulley 21 moves in the first direction D1. When the user moves the operation lever 10 to the right, the shaft member 22 or the pulley 21 moves in the second direction D2.

The pair of wires 6 connected to the pulley 21 is pulled and relaxed, by rotation of the pulley 21 about the central axis. For example, when the pulley 21 rotates in one direction about the central axis, one of the paired wires 6 is pulled and, the other is relaxed. In response to pulling and relaxing of the pair of wires 6, the bending portion 2b bends and deforms as described above. In this manner, according to the endoscope 1 of the present embodiment, when the user moves the operation lever 10 in the vertical direction along the first axis A1, the bending portion 2b bends and deforms.

The holding portion 30 and the driving portion 40 are disposed in the internal space 3c of the operation section 3. The optical fiber cable 7 is inserted into the internal space 3c through the through hole 3d from the outside of the operation section 3. As described above, the optical fiber cable 7 is inserted into the channel 5 through the second opening 5b. Note that although not illustrated, a seal member is disposed in the channel 5. The seal member allows a movement of the optical fiber cable 7 along the longitudinal axis, and seals a gap between an inner wall of the channel 5 and the optical fiber cable 7. The seal member is, for example, an O-shaped ring. The seal member prevents fluid that has entered the channel 5 from the first opening 5a from entering the internal space 3c.

The holding portion 30 holds the optical fiber cable 7 in the internal space 3c. The driving portion 40 configuring a driving member moves the holding portion 30 in a direction along a longitudinal axis of the optical fiber cable 7, in response to a movement of the operation lever 10 along the second axis A2. The driving portion 40 includes a mechanism configured to convert a movement of the shaft member 22 in a direction parallel to the central axis into a movement of the holding portion 30 in the direction along the longitudinal axis of the optical fiber cable 7.

In the present embodiment, the driving portion 40 includes a first arm portion 41 and a second arm portion 42 that swing about the support shaft 43 in the internal space 3c, for example. The support shaft 43 is disposed at a position spaced away from the central axis of the shaft member 22 in a radial direction. The support shaft 43 is substantially parallel to a plane orthogonal to the central axis of the shaft member 22.

The first arm portion 41 and the second arm portion 42 extend in different radial directions from the support shaft 43. The second arm portion 42 is fixed to the first arm portion 41. Accordingly, both the first arm portion 41 and the second arm portion 42 swing about the support shaft 43.

Note that the structure for swinging the first arm portion 41 and the second arm portion 42 is not limited to the structure using the support shaft 43. For example, the first arm portion 41 and the second arm portion 42 may be supported by an elastically deformable plate-spring-shaped member, and swing in response to deformation of the plate-spring-shaped member.

The first arm portion 41 extends from the support shaft 43 toward the central axis of the shaft member 22. The first arm portion 41 is disposed so as to cross a trajectory of a movement of the distal end portion 22b of the shaft member 22. As illustrated in FIG. 4, when the shaft member 22 is located at the end of the movable range in the first direction D1, the first arm portion 41 is spaced apart from the distal end portion 22b of the shaft member 22 by a predetermined distance. Then, when the shaft member 22 pushes away the stopper 24 and moves in the second direction D2 by a predetermined distance or greater, the distal end portion 22b of the shaft member 22 comes into contact with the first arm portion 41.

The driving portion 40 includes an urging portion 44 that urges the first arm portion 41 in such a direction that the first arm portion 41 is pressed against the distal end portion 22b of the shaft member 22. More specifically, when the distal end portion 22b of the shaft member 22 is in contact with the first arm portion 41, the urging portion 44 generates a force for urging the shaft member 22 in the first direction D1.

Note that the urging portion 44 of the present embodiment is a plate-spring-shaped member protruding from the inner wall surface of the internal space 3c of the operation section 3. In the present embodiment, the urging portion 44 is integrally molded with a resin member configuring the inner wall surface of the internal space 3c.

When the distal end portion 22b of the shaft member 22 is in contact with the first arm portion 41, the first arm portion 41 is swung about the support shaft 43 in accordance with a movement of the shaft member 22 parallel to the central axis, by an urging force of the urging portion 44.

The holding portion 30 is disposed at a distal end portion 42a of the second arm portion 42. The second arm portion 42 is disposed such that a distance between the distal end portion 42a and the second opening 5b changes when the second arm portion 42 swings about the support shaft 43. The second arm portion 42 is disposed at such a position that the distal end portion 42a faces the second opening 5b.

The distance between the holding portion 30 and the second opening 5b changes when the second arm portion 42 swings. In the present embodiment, a tangent line to a movement trajectory of the holding portion 30 passes near the second opening 5b. Accordingly, when the second arm portion 42 swings about the support shaft 43, the holding portion 30 moves in a direction substantially along the longitudinal axis of the optical fiber cable 7 extending out of the second opening 5b.

As described above, the second arm portion 42 in which the holding portion 30 is disposed swings about the support shaft 43, together with the first arm portion 41. The first arm portion 41 swings about the support shaft 43 in accordance with a movement of the shaft member 22 in the first direction D1 and the second direction D2.

In the present embodiment, when the shaft member 22 moves in the second direction D2, the distance between the holding portion 30 and the second opening 5b is reduced. When the shaft member 22 moves in the first direction D1, the distance between the holding portion 30 and the second opening 5b is increased.

According to the endoscope 1 of the present embodiment having the configuration described above, when the distal end portion 10a of the operation lever 10 moves along the second axis A2, the shaft member 22 moves in the first direction D1 or the second direction D2 parallel to the central axis. When the shaft member 22 moves in the first direction D1 or the second direction D2, the holding portion 30 moves in the direction substantially along the longitudinal axis of the optical fiber cable 7. Therefore, according to the endoscope 1 of the present embodiment, when the distal end portion 10a of the operation lever 10 moves in the horizontal direction along the second axis A2, the optical fiber cable 7 inserted in the channel 5 advances or retracts along the longitudinal axis.

More specifically, when the distal end portion 10a of the operation lever 10 moves rightward, the optical fiber cable 7 moves to the distal end 7a side. When the distal end portion 10a of the operation lever 10 moves leftward, the optical fiber cable 7 moves to the proximal end side. Accordingly, the urging portion 44 generates an urging force that urges the holding portion 30 in such a direction that the optical fiber cable 7 moves to the proximal end side.

The following describes a method of operating the endoscope 1 of the present embodiment when performing a treatment for removing a urinary stone in a subject.

First, before inserting the insertion section 2 into a subject, the user moves the operation lever 10 to the left end of the movable range such that the shaft member 22 is located at the end of the movable range in the first direction D1. With this operation, the shaft member 22 is locked at the end of the movable range in the first direction D1 by the stopper 24.

Subsequently, the user adjusts the position where the optical fiber cable 7 is held by the holding portion 30. With this adjustment, the distal end 7a of the optical fiber cable 7 is located on the inner side of the first opening 5a of the channel 5. Note that, in the endoscope 1, at the time of manufacturing, the optical fiber cable 7 may be fixed to the holding portion 30 so as to be located at the above-described position. In this case, the user does not have to make this adjustment.

Then, the user inserts the insertion section 2 into the subject. When inserting the insertion section 2 into the subject, the user performs a bending operation of bending and deforming the bending portion 2b. The user performs the bending operation by moving the operation lever 10 in the vertical direction along the first axis A1.

The shaft member 22 is locked at the end in the first direction D1 by the stopper 24, and is spaced away from the driving portion 40 by a predetermined distance. The holding portion 30 is urged by the urging portion 44 in such a direction that the optical fiber cable 7 moves to the proximal end side. Accordingly, as long as the user does not intentionally apply a force for moving the operation lever 10 rightward, the distal end 7a of the optical fiber cable 7 is held in the channel 5.

After the insertion section 2 reaches a part where a stone can be observed, the user moves the operation lever 10 rightward by a predetermined distance or greater such that the distal end 7a of the optical fiber cable 7 protrudes from the first opening 5a.

After that, the user can perform a bending operation of the bending portion 2b by moving the operation lever 10 in the vertical direction with a thumb of one hand holding the operation section 3. The user can also advance and retract the optical fiber cable 7, and change the amount by which the optical fiber cable 7 protrudes from the first opening 5a, by moving the operation lever 10 in the horizontal direction with the thumb of the hand holding the operation section 3.

With use of the endoscope 1 of the present embodiment, the user can perform a bending operation of the insertion section 2, an operation of rotating the insertion section 2 about the longitudinal axis, and an operation of advancing and retracting the optical fiber cable 7 at the same time, using one hand holding the operation section 3. Therefore, with use of the endoscope 1 of the present embodiment, the user can advance and retract both the insertion section 2 and the optical fiber cable 7 at the same time, by advancing and retracting the insertion section 2 using the other hand not holding the operation section 3. Therefore, the user can move the distal end 7a of the optical fiber cable 7 to a position of irradiating a stone with laser light in the subject in a short time period.

As described above, according to the endoscope 1 of the present embodiment, it is possible to perform an operation of moving the optical fiber cable 7 in the subject in a short time period.

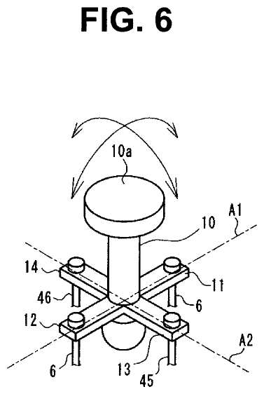

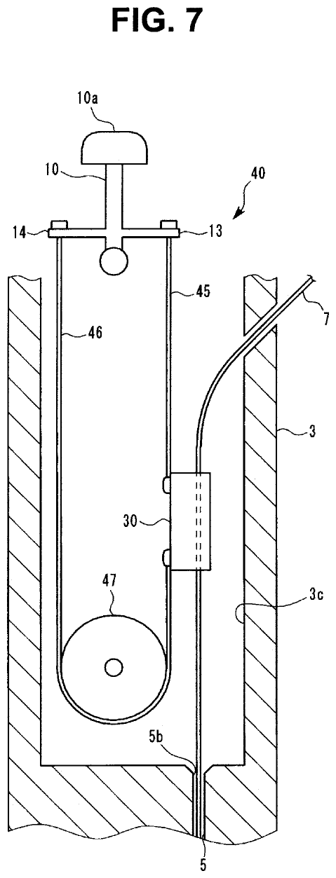

FIGS. 6 and 7 illustrate a modification of the endoscope 1 of the present embodiment. As illustrated in FIG. 6, an endoscope 1 of the present modification includes an operation lever 10 in the form of a so-called joystick that tilts in all directions.

A first arm portion 11, a second arm portion 12, a third arm portion 13, and a fourth arm portion 14 extend from the operation lever 10 in directions orthogonal to a central axis of the operation lever 10. The first arm portion 11 and the second arm portion 12 extend in directions opposite to each other along a first axis A1. The third arm portion 13 and the fourth arm portion 14 extend in directions opposite to each other along a second axis A2. The first axis A1 and the second axis A2 are orthogonal.

Wires 6 are connected to respective distal end portions of the first arm portion 11 and the second arm portion 12. The pair of wires 6 is arranged such that the longitudinal axis is substantially orthogonal to the first arm portion 11 and the second arm portion 12 at a connection portion between the first arm portion 11 and the second arm portion 12.

In the present modification, when the operation lever 10 tilts in the direction along the first axis A1, the pair of wires 6 is pulled and relaxed. Accordingly, according to the endoscope 1 of the present modification, the bending portion 2b bends and deforms in response to tilting of the operation lever 10 in a direction along the first axis A1.

Proximal ends of a first wire 45 and a second wire 46 are connected to distal end portions of the third arm portion 13 and the fourth arm portion 14. As illustrated in FIG. 7, the first wire 45 and the second wire 46 configure a driving portion 40. The driving portion 40 of the present modification includes the first wire 45, the second wire 46, and a pulley 47.

Distal ends of the first wire 45 and the second wire 46 are connected to a holding portion 30 in an internal space 3c of an operation section 3. The pulley 47 is disposed in the internal space 3c of the operation section 3. The second wire 46 is wound around the pulley 47 so that the direction is changed by 180 degrees.

In the present modification, the first wire 45 and the second wire 46 are arranged to form a loop. The first wire 45 and the second wire 46 advance and retract in the direction along the longitudinal axis in response to tilting of the operation lever 10 in a direction along the second axis A2. Then, the holding portion 30 moves in accordance with an advancement movement and a retraction movement of the first wire 45 and the second wire 46 in the direction along the longitudinal axis, so that a distance between the holding portion 30 and a second opening 5b changes.

As described above, according to the endoscope 1 of the present modification, it is possible to perform a bending operation of the bending portion 2b and an operation of advancing and retracting the optical fiber cable 7, by changing the direction and angle of tilt of the operation lever 10.

What is claimed is:

1. An endoscope comprising:
an insertion section configured to be inserted into a subject, and including a bending portion configured to bend in a predetermined direction in response to pulling and relaxing of a pair of wires;
an operation section connected to a proximal end side of the insertion section;
a pulling mechanism to which the pair of wires is connected, the pulling mechanism being disposed at the operation section and configured to pull and relax the pair of wires;
a holding portion disposed at the operation section, and holding a treatment member inserted in a channel provided inside the insertion section; and
a driving member disposed at the operation section, and configured to move the holding portion in a direction along a longitudinal axis of the treatment member,
wherein the pulling mechanism includes
a pulley to which the pair of wires is connected,
a shaft member protruding from the pulley, and having a central axis coinciding with a central axis of the pulley, and
a support portion supporting the shaft member such that the shaft member is rotatable about the central axis, and configured to allow a movement of the shaft member in a first direction and a second direction that are parallel to the central axis of the shaft member.

2. The endoscope according to claim 1, further comprising:
an operation lever disposed at the operation section, and configured to move relative to the operation section along a first axis and a second axis that intersects the first axis,
wherein the pulling mechanism moves the holding portion in a direction along the longitudinal axis of the treatment member in response to a movement of the operation lever along the second axis, and
the operation lever is coupled to the shaft member or the pulley, and is configured to move along the first axis when the operation lever rotates about the central axis with respect to the operation section, and move along the second axis when the operation lever moves parallel to the central axis.

3. The endoscope according to claim 1, wherein the treatment member is an optical fiber cable.

4. The endoscope according to claim 1,
wherein the driving member is configured to transmit a movement of the shaft member in the first direction and the second direction to the holding portion when the driving member is in contact with the shaft member, and
the shaft member is spaced apart from the driving portion by a predetermined distance when the shaft member is located at an end in the first direction in a movable range in a direction parallel to the central axis.

5. The endoscope according to claim 4, wherein the driving member includes a mechanism configured to convert a movement of the shaft member in the direction parallel to the central axis into a movement of the holding portion in the direction along the longitudinal axis of the treatment member.

6. The endoscope according to claim 5,
wherein the driving member includes
a support shaft, and
a first arm portion and a second arm portion extending from the support shaft in different radial directions, and configured to swing about the support shaft,
the channel allows communication between a first opening provided in the insertion section and a second opening provided in the operation section,
the first arm portion extends from the support shaft toward the central axis of the shaft member, and is configured to swing about the support shaft in accordance with a movement of the shaft member parallel to the central axis when the first arm portion comes into contact with the shaft member, and
the holding portion is disposed at the second arm portion such that the treatment member advances and retracts in the direction along the longitudinal axis when a distance between the holding portion and the second opening is changed by swinging of the second arm portion.

7. The endoscope according to claim 5,
wherein the driving member includes
a plate-spring-shaped member that is elastically deformable, and
a first arm portion and a second arm portion supported by the plate-spring-shaped member, the first arm portion and the second arm portion being configured to swing when the plate-spring-shaped member deforms.

8. The endoscope according to claim 4,
wherein the support portion includes a stopper configured to lock the shaft member at the end in the first direction in the movable range in the direction parallel to the central axis, and
the stopper is configured to allow a movement of the shaft member in the second direction when a force in the second direction applied to the shaft member exceeds a predetermined value.

9. The endoscope according to claim 1, further comprising:
an urging portion configured to urge the holding portion in a direction toward a proximal end of the treatment member.

10. The endoscope according to claim 1,
wherein the channel includes
a first opening provided in a distal end portion of the insertion section,
a second opening provided in the operation section, and configured to allow insertion of the treatment member held by the holding portion, and
a third opening provided in the operation section, and configured to allow insertion of a treatment instrument into and removal of the treatment instrument from the channel.
